(19) United States
(12) Patent Application Publication
(10) Pub. No.: US 2006/0047224 A1
(43) Pub. Date: Mar. 2, 2006

(54) Polymer coated guide wire

(76) Inventor: Ryan Grandfield, Murrieta, CA (US)

Correspondence Address: FULWIDER PATTON, 6060 CENTER DRIVE, 10TH FLOOR, LOS ANGELES, CA, 90045, US

(21) Appl. No.: 10/931,746

(22) Filed: Sep. 1, 2004

(51) Int. Cl. A61M 25/00

(52) U.S. Cl. 600/585

(57) ABSTRACT

A guide wire having different polymers applied to different components thereof so as to differentially modify the performance characteristics of such components in order to achieve a desired performance behavior for such guide wire. A guide wire having a tapered distal coil coated with a highly conforming polymer while sections proximal thereto are coated with a substantially less conforming polymer will yield a guide wire that is more easily advanced through vasculature while nonetheless providing tactile feedback.

### BACKGROUND OF THE INVENTION

This invention relates to the field of guide wires for advancing intraluminal devices such as stent delivery catheters, balloon dilatation catheters, atherectomy catheters and the like within a patient's body, specifically, within a patient's vasculature.

In a typical percutaneous procedure in a patient's coronary system, a guiding catheter having a preformed distal tip is percutaneously introduced into a patient's peripheral artery, e.g. femoral, radial or brachial artery, by means of a conventional Seldinger technique and advanced therein until the distal tip of the guiding catheter is seated in the ostium of a desired coronary artery. There are two basic techniques for advancing a guide wire into the desired location within the patient's coronary anatomy, the first is a preload technique which is used primarily for over-the-wire (OTW) devices and the bare wire technique which is used primarily for rail type systems. With the preload technique, a guide wire is positioned within an inner lumen of an OTW device such as a dilatation catheter or stent delivery catheter with the distal tip of the guide wire just proximal to the distal tip of the catheter and then both are advanced through the guiding catheter to the distal end thereof. The guide wire is first advanced out of the distal end of the guiding catheter into the patient's coronary vasculature until the distal end of the guide wire crosses the arterial location where the interventional procedure is to be performed, e.g. a lesion to be dilated or a dilated region where a stent is to be deployed.

The catheter, which is slidably mounted onto the guide wire, is advanced out of the guiding catheter into the patient's coronary anatomy over the previously introduced guide wire until the operative portion of the intravascular device, e.g. the balloon of a dilatation or a stent delivery catheter, is positioned across the arterial location. Once the catheter is in position with the operative means located within the desired arterial location, the interventional procedure is performed. The catheter can then be removed from the patient over the guide wire. Usually, the guide wire is left in place for a period of time after the procedure is completed to ensure reaccess to the arterial location if it is necessary. For example, in the event of arterial blockage due to dissected lining collapse, a rapid exchange type perfusion balloon catheter such as described and claimed in U.S. Pat. No. 5,516,336 (McInnes et al.), can be advanced over the in-place guide wire so that the balloon can be inflated to open up the arterial passageway and allow blood to perfuse through the distal section of the catheter to a distal location until the dissection is reattached to the arterial wall by natural healing.

With the bare wire technique, the guide wire is first advanced by itself through the guiding catheter until the distal tip of the guide wire extends beyond the arterial location where the procedure is to be performed. Then a rail type catheter, such as described in U.S. Pat. No. 5,061,273 (Yock) and the previously discussed McInnes et al. which are incorporated herein by reference, is mounted onto the proximal portion of the guide wire which extends out of the proximal end of the guiding catheter which is outside of the patient. The catheter is advanced over the guide wire, while the position of the guide wire is fixed, until the operative means on the rail type catheter is disposed within the arterial location where the procedure is to be performed. After the procedure the intravascular device may be withdrawn from the patient over the guide wire or the guide wire advanced further within the coronary anatomy for an additional procedure.

Conventional guide wires for angioplasty, stent delivery, atherectomy and other vascular procedures usually comprise an elongated core member with one or more tapered sections near the distal end thereof and a flexible body such as a helical coil or a tubular body of polymeric material disposed about the distal portion of the core member. A shapeable member, which may be the distal extremity of the core member or a separate shaping ribbon which is secured to the distal extremity of the core member, extends through the flexible body and is secured to the distal end of the flexible body by soldering, brazing or welding which forms a rounded distal tip. Torquing means are provided on the proximal end of the core member to rotate, and thereby steer, the guide wire while it is being advanced through a patient's vascular system.

Specially configured guide wires have been proposed for facilitating the crossing of chronic total occlusions (CTO) or highly occluded sections of vessel. Use of tapered distal tips have been suggested for more easily penetrating the occlusion wherein a variety of structures can be relied upon to provide a strong yet flexible taper. Although a coiled structure delivers strength and flexibility, the coils can be susceptible to becoming snagged on occlusive material and can potentially be subject to being pulled apart upon retraction of the guide wire.

Further details of guide wires, and devices associated therewith for various interventional procedures can be found in U.S. Pat. No. 4,748,986 (Morrison et al.); U.S. Pat. No. 4,538,622 (Samson et al.): U.S. Pat. No. 5,135,503 (Abrams); U.S. Pat. No. 5,341,818 (Abrams et al.); U.S. Pat. No. 5,345,945 (Hodgson, et al.) and U.S. Pat. No. 5,636,641 (Fariabi) which are hereby incorporated herein in their entirety by reference thereto.

While it is most desirable to minimize the force necessary for advancing the guide wire through vasculature it is nonetheless desirable to retain sufficient tactile feedback in order to allow the clinician to feel wire movement. These two performance criteria tend to compete with one another to the extent that an increase in lubricity would typically be expected to result in a decrease in tactile feedback and vice versa. While lubricity and tactile feedback are desirable performance attributes for substantially any guide wire application including cardiovascular, peripheral and carotid procedures, it is especially desirable in total occlusion cases when the guide wire must be sufficiently lubricious to penetrate and navigate tiny micro-channels in the lumen, but nonetheless allow the clinician to feel wire movement. A guide wire is needed that is simultaneously capable of satisfying these performance requirements.

### SUMMARY OF THE INVENTION

The guide wire of the present invention overcomes the shortcomings of previously known guide wire configurations to the extent that the device can easily be advanced through vasculature yet provide a high degree of tactile feedback. The guide wire is suitable for use in cardiovascular, peripheral or carotid procedures and is especially well adapted for use in total occlusion cases. The desired performance characteristics are achieved with the application of a polymer to the distal tip of the guide wire that is different than the polymer that is used to coat sections proximal thereto.

In a preferred embodiment, a very thin, highly shape conforming polymer is used to coat the tip coil while a thicker, less conforming polymer is applied proximal thereto. The high conformance of the polymer to the tip coil causes the polymer surface to assume the ribbed shape of the underlying coil. The reduced conformance of the polymer coating proximal to the tip coil serves to present a smoother surface for contacting the vasculature through which it is advanced to thereby greatly reduce drag and resistance. The polymer coatings provide for a lubricious surface to reduce drag and friction while the ribbed surface at the distal tip of the guide wire provides the desired tactile feedback. Additionally, the presence of a polymer between and/or over the coils prevents the coils from becoming snagged on occlusive material and being pulled apart upon retraction of the guide wire.

Specific polymer combinations can be selected from a large variety of polymers so as to achieve the desired performance characteristics of the guide wire of the present invention. Suitable polymers include, but are not limited to, urethanes, nylons, PTFE, and FEP. A polymer that is selected for application to a specific portion of the guide wire may also be loaded with a radiopaque material, such as tungsten, in order to impart a desired degree of lubricity or a desired “feel” to the guide wire in addition to enabling that portion of the guide wire to be visualized during a procedure.

The selected combination of polymers can be applied using any of variety of techniques as well as a combination thereof. For example, the guide wire can be dip coated to not only form a polymeric outer surface but to additionally penetrate between coils and fill the space between the coils and an underlying core section. Alternatively, the polymer can be applied in the form of heat shrink tubing or can be extruded there onto. As a further alternative, a coil wire can be coated with the polymer prior to being coiled. The various polymer coatings can be applied so as to overlap, form a butt joint or define a gap there between.

The guide wire of the present invention may be based on any of various guide wire platforms which may in turn incorporate any of the various guide wire components including core tapers, tip flats, and core materials, while tip coil configurations can varied to meet specific design requirement. Typically a guide wire would have an elongate core member with proximal and distal core sections and a flexible tubular body such as a helical coil or polymeric body disposed about and secured to the distal section of the core member. The flexible tubular body such as a helical coil is secured by its distal end to the distal tip of the distal core section or to the distal tip of a shaping ribbon secured to the distal core section in a conventional fashion. The helical coil may be secured at its distal end by application of an adhesive or epoxy, soldering, brazing or welding to form a rounded distal tip to the guiding member as done with commercially available guide wire for procedures within a patient's coronary artery. A tapered tip coil may additionally be fitted for CTO applications.

These and other advantages of the invention will become apparent from the following detailed description of preferred embodiments which, taken in conjunction with the drawings, illustrate by way of example the principles of the invention.

### BRIEF DESCRIPTION OF THE DRAWINGS

FIG. 1 is a side view of a distal section of a guide wire of the present invention;

FIG. 2 is an enlarged view of the circled section 2 of FIG. 1;

FIG. 3 is a view similar to FIG. 2 of an alternative embodiment; and

FIG. 4 is a view similar to FIG. 2 of another alternative embodiment of the guide wire of the present invention.

### DETAILED DESCRIPTION OF PREFERRED EMBODIMENTS

The Figures depict various embodiments of the guide wire of the present invention wherein different polymers are applied to selected components of a guide wire in order to modify the performance characteristics of the underlying components. A guide wire can thereby be configured to simultaneously provide for high degree of tactile feedback while being highly lubricious to thereby minimize drag and friction.

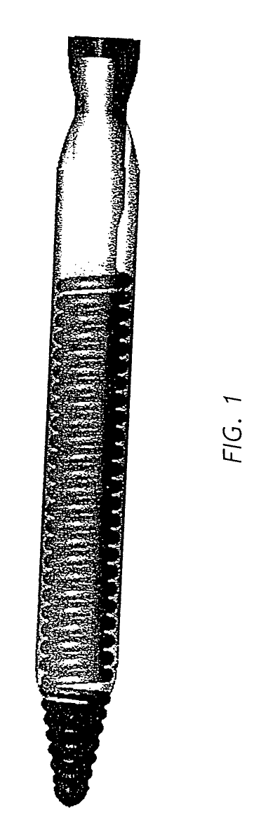

FIG. 1 is a side view of the distal section of guide wire 12 of the present invention that is especially well adapted for use in CTO applications. A tapered distal tip coil 16 is attached to a section of coil of constant diameter 14 which in turn is attached to a core 17 that extends proximally therefrom. A first polymer coating 18 is applied to the distal tip coil wherein such polymer is selected for its ability to conform to an underlying surface and thereby substantially assume the rippled shape of the underlying coil's outer surface. A second different polymer 20 is applied to the coil of constant diameter. Such second polymer is selected for its non conforming properties wherein a substantially smooth outer surface results despite the rippled shape of the exterior surface of the underlying coiled structure. In a preferred embodiment, the highly conforming first polymer extends from the distal tip of the tip coil proximally therefrom while the second polymer extends proximally from a point located approximately 2.5 centimeters form the distal tip.

FIG. 2 is an enlarged view of the circled section shown in FIG. 1. This view shows an overlapping configuration of the two polymers 18, 20. The highly conforming first polymer 18 is shown extending from the distal end of the tip coil 16 proximally, while the relatively non-conforming second polymer 20 extends proximally from a point proximal to the proximal end of the distal tip. As result the outer surface of the guide wire has a rippled shape in the tapered section while being smooth proximal thereto. The distal end 22 of the second polymer can substantially coincide with the proximal end of the tip coil or can be positioned along the tip coil or alternatively, at a point along the coil of constant diameter. The overlap between the two polymers may be selected to define a section just a few millimeters in length to a section extending along the entire length of the second polymer.

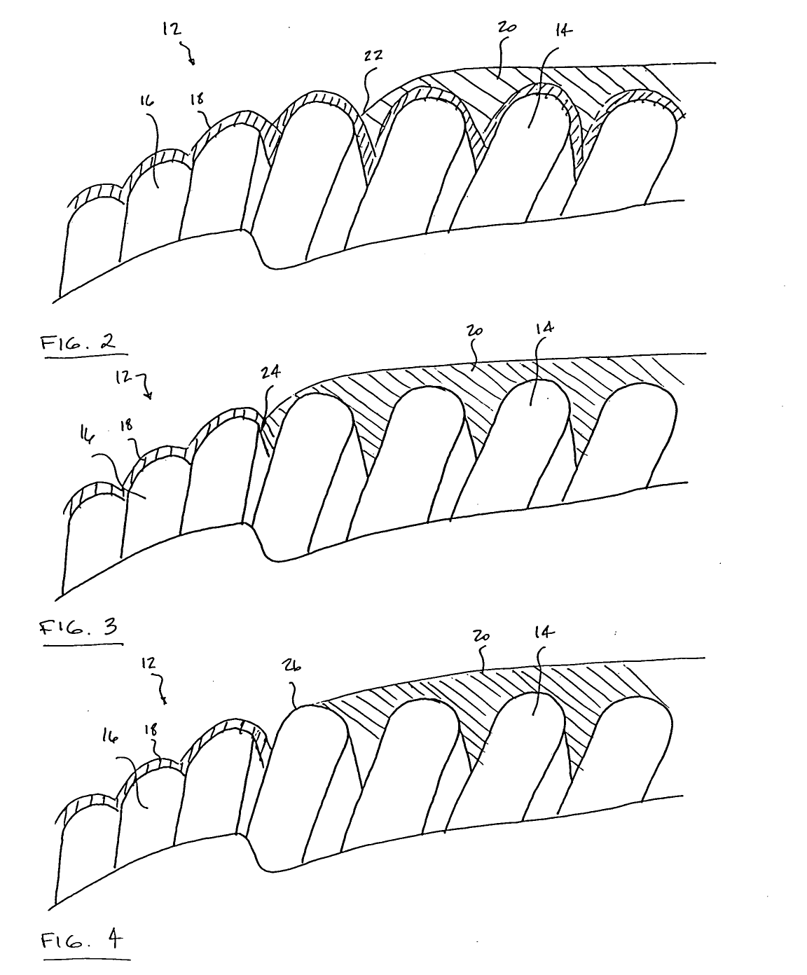

FIG. 3 is a view similar to FIG. 2 of an alternative embodiment of the guide wire configuration shown in FIG. 2, wherein the first and second polymers 18, 20 form a butt joint 24. The highly conforming first polymer 18 is shown extending proximally from the distal end of the tip coil 16 to a point approximately coinciding with the proximal end of the tip coil. The non conforming second polymer 20 butts up against the proximal end of the first polymer and extends proximally therefrom. Such configuration again provides a structure that includes a distal tip with a rippled surface while a smooth surface extends proximally therefrom. The butt joint can be positioned to coincide with the proximal end of the tapered distal coil, can be positioned along the length of the distal coil or can be positioned along the length of the coil of constant diameter.

FIG. 4 is view similar to FIG. 2 of another alternative embodiment of the guide wire shown in FIG. 2 wherein the proximal end of the first polymer 18 and the distal end of the second polymer 20 are spaced apart from one another so as to define a gap 26. In the particular embodiment that is shown, the gap is positioned just proximal to the proximal end of the distal coil. Such configuration again provides a structure that includes a distal tip with a rippled surface while a smooth surface extends proximally therefrom. The gap can be positioned to coincide with the proximal end of the tapered distal coil, can be positioned along the length of the distal coil or can be positioned along the length of the coil of constant diameter.

A wide variety of polymers can be employed in the practice of the present invention including, but not limited to, urethanes, nylons, PTFE or FEP. The polymer selected for application to the distal most section of the guide wire is selected for its ability to conform to the underlying shape wherein properties such low viscosity and the ability to form a thin layer are critical. The low viscosity also allows the polymer to fill the available space in the interior of the coils as well as bridging the space between adjacent coils. UV curable aliphatic urethane acrylate has been found to be especially well suited for such application. The second polymer is selected for its ability to be applied in a relatively thick layer and for having a sufficiently high viscosity to form a smooth outer surface despite a rippled substrate. A polyurethane ether polymer has been found to be especially well suited for such application.

The polymers applied to the guide wire can be rendered radiopaque in order to enable visualization of the guide wire during a procedure. Either or both of the polymers used to coat the selected sections of the guide wire of the present invention can be loaded with high levels of tungsten in order to achieve the desired degree of visibility.

The selected polymers can be applied to the guide using any of a wide variety of methods, including, but not limited to dip coating, spray coating, extrusion, necking or heat shrinking. Well know masking methods can be used to control the application of a polymer to selected portions of the guide wire. The selected polymers can be applied to the coiled structures or the coiled structures can be formed from polymer coated wire.

In use, the guide wire of the present invention offers a desirable degree of tactile feedback to the clinician while simultaneously minimizing drag and friction. The ripples on the leading surfaces of the guide wire serve to briefly engage occlusive material to provide clarity of wire movement while the lubricity of both polymers minimizes drag as the guide wire is advanced especially when negotiating tortuous paths. Additionally, the polymers serve to lock the coils together to prevent them from becoming snagged or from pulling apart as the guide wire is retracted.

While particular forms of the invention have been described and illustrated, it will also be apparent to those skilled in the art that various modifications can be made without departing form the spirit and scope of the invention. Accordingly, it is not intended that the invention be limited except by the appended claims.

What is claimed is:

1. A guide wire, comprising:
a distal section having a coiled structure wherein its outer surface has a rippled shape;
a first polymer having high conformance properties applied to a distal portion of said coiled structure wherein its outer surface substantially assumes the rippled shape of the outer surface of the underlying coiled structure; and
a second polymer having a relatively low conformance properties applied to a portion of said coiled structure proximal to said distal portion wherein its outer surface does not assume the rippled shape of the underlying coiled structure so as to be substantially smooth.

2. The guide wire of claim 1, wherein said first and second polymers overlap one another such that the proximal end of said first polymer is proximal to the distal end of the second polymer.

3. The guide wire of claim 1, wherein the proximal end of said first polymer and the distal end of said second polymer are butted up against one another.

4. The guide wire of claim 1, wherein said first and second polymers are spaced so as to define a gap between the proximal end of said first polymer and the distal end of said second polymer.

5. The guide wire of claim 1, wherein said first polymer comprises a UV curable aliphatic urethane acrylate.

6. The guide wire of claim 1, wherein said second polymer comprises a polyurethane ether polymer.

7. The guide wire of claim 1, wherein said first polymer is radiopaque.

8. The guide wire of claim 1, wherein said second polymer is radiopaque.

9. The guide wire of claim 1, wherein said distal section of said guide wire includes a tapered tip coil and a coil of constant diameter proximal thereto.

10. The guide wire of claim 9, wherein said tapered tip coil substantially comprises said distal portion of said distal section.

11. The guide wire of claim 1, wherein said first polymer is applied by dip coating.

12. The guide wire of claim 1, wherein at least one of said polymers is heat shrunk onto said coiled structure.

13. The guide wire of claim 12, wherein both of said polymers are heat shrunk onto said coiled structure.

14. The guide wire of claim 1, wherein said first polymer is applied to wire before such wire is wound to form said coil structure.

15. The guide wire of claim 9, wherein said first polymer is applied to wire before such wire is would to form said tapered tip coil.

16. A guide wire, comprising:
a tapered tip coil having an outer surface with a rippled shape;
a coil of constant diameter having outer surface with a rippled shape disposed proximally to said tapered tip coil;
a high conformance first polymer disposed about said tip coil wherein its outer surface substantially assumes said rippled shape of the outer surface of said tip coil; and
a low conformance second polymer disposed about said coil of constant diameter wherein its outer surface is substantially smooth.

17. The guide wire of claim 16, wherein said first polymer comprises a UV curable aliphatic urethane acrylate.

18. The guide wire of claim 16, wherein said second polymer comprises a polyurethane ether polymer.
